(19) United States
(12) Patent Application Publication
(10) Pub. No.: US 2020/0010385 A1
(43) Pub. Date: Jan. 9, 2020

(54) PROCESS FOR REMOVING ALKENE AND/OR ALKYNE FROM A HYDROCARBON FEEDSTOCK

(71) Applicant: SCG Chemicals Co., Ltd., Bangkok (TH)

(72) Inventor: Kongkiat SURIYE, Bangkok (TH)

(73) Assignee: SCG Chemicals Co., Ltd., Bangkok (TH)

(21) Appl. No.: 16/468,710

(22) PCT Filed: Nov. 20, 2017
(86) PCT No.: PCT/EP2017/079790

§ 371 Date: Jun. 12, 2019

(30) Foreign Application Priority Data
Dec. 13, 2016 (EP) 16203601.6

(51) Int. Cl. C07C 7/12; B01J 20/12; B01J 20/34

(52) U.S. Cl. CPC C07C 7/12; B01J 20/3458; B01J 20/12

(57) ABSTRACT

The present invention relates to a process for purifying a hydrocarbon feedstock, said process comprising the steps: (a) providing the hydrocarbon feedstock comprising an aromatic compound and at least one compound, selected from the group consisting of alkene, alkyne, nitrogen-containing compound or mixtures thereof; and (b) contacting the hydrocarbon feedstock with an acidic montmorillonite adsorbent at a temperature in the range from 10 to 60° C.

The present invention relates to a process for removing alkene and/or alkyne from a hydrocarbon feedstock comprising an aromatic compound.

It is known that there are various kinds of mixtures of substance containing unsaturated hydrocarbon. Unsaturated hydrocarbon contributes to the bromine index (BI) which is an analytical parameter in order to control the quality of aromatic compound and can be obtained by the amount of bromine in milligrams adsorbed by 100 grams of the sample under given conditions. A low value of BI is preferable to place an aromatic product in the commercial market. Moreover, lower amount of contamination by alkene and/or alkyne or other unsaturated non-aromatic hydrocarbon is normally desired because they could be harmful for a next downstream process utilizing the aromatic compound and can cause undesirable by-products.

It is well known that removing unsaturated non-aromatic hydrocarbon from an aromatic compound may be based on alkylation reaction. Most of prior research and development were focused on an alkylation catalyst starting from activated clay to advanced zeolite.

Such an alkylation reaction causes loss of valuable aromatic compound because it involves reacting the aromatic compound with alkene and/or alkyne to form alkyl aromatic which can normally be easily removed from the aromatic compound by simple distillation.

The clay type catalyst used in the alkylation reaction could not be effectively regenerated because heavy hydrocarbon could be converted to hard coke which is hard to remove at mild temperature resulting in high operation cost. Moreover, a complete removal which has to be performed at high temperature will finally destroy the crystal structure of clay.

U.S. Pat. No. 3,485,884 discloses a method for treating an aromatic hydrocarbon containing mixture by contacting the mixture (contaminated with olefins and sulfur compounds) with aluminum silicate at an elevated temperature within the range of about 200 to about 260° C. followed by distillation. The contacting condition causes alkylation reaction.

U.S. Pat. No. 6,368,496 discloses a method for removing bromine reactive hydrocarbon contaminants from an aromatic stream by providing the aromatic stream having a negligible diene level. The feed stream is then contacted with an active catalyst at conditions including a moderately elevated temperature ranging from about 93 to 260° C. under alkylation reaction to remove unsaturated hydrocarbon.

WO 2011/008344 A1 discloses a method for removing colored-impurities and sulfur-impurities which are bromine index contributors from a hydrocarbon stream by contacting the hydrocarbon stream with an adsorbent that has been pretreated to get a pH about 10.

It is therefore an object of the present invention to provide an improved process for removing alkene and/or alkyne from a hydrocarbon feedstock overcoming drawbacks of the prior art, in particular allowing high efficiency in hydrocarbon purification and consequently reduction of BI value where regeneration is enabled and loss of desirable hydrocarbon is avoided.

The object has been achieved by a process for purifying a hydrocarbon feedstock, said process comprising the steps of providing the hydrocarbon feedstock comprising an aromatic compound, and at least one compound selected from the group consisting of alkene, alkyne, nitrogen-containing compound or mixtures thereof; and contacting the hydrocarbon feedstock with an acidic montmorillonite adsorbent at a temperature in the range from 10 to 60° C., preferably 15 to 35° C.

While most of terms used herein will be recognizable to those of ordinary skill in the art, it should be understood, however, that when not explicitly defined, terms should be interpreted as adopting a meaning presently accepted by those of ordinary skill in the art. Definition and/or interpretations should not be incorporated from other patent applications, patents, or publications, related or not, unless specifically stated in this specification or if the incorporation is necessary for maintaining validity.

As used herein, the term “hydrocarbon feedstock” refers to an unprocessed stream used to be supplied to a manufacturing process comprising substantially of hydrocarbon, wherein hydrocarbon is any molecule consisting entirely of hydrogen and carbon which could be saturated hydrocarbon, unsaturated hydrocarbon, or aromatic hydrocarbon.

The term “aromatic compound” refers to substances that consist of one or more rings that contain alternating single and double bonds in its chemical structure. In one embodiment, the aromatic compound is an aromatic hydrocarbon compound which preferably consists of one aromatic ring. In one embodiment, the aromatic compound is an aromatic hydrocarbon compound selected from benzene, toluene, xylene, ethyl benzene, their derivatives, and mixtures thereof, more preferably benzene, toluene, and a mixture thereof. In another representative embodiment, the aromatic compound is linear alkylbenzene. The linear alkylbenzene refers to a family of organic compounds generally produced by alkylation of benzene with a C10-C16 linear paraffin, and therefore has a chemical formula C₆H₅CₙH₂ₙ₊₁, wherein n is typically between 10 and 16, more often between 10 and 13.

The term “alkene” refers to any hydrocarbon molecule containing at least one double carbon-carbon bond which can be of aliphatic or cyclic structure. Typically for the present invention, C4 to C10 alkene may be present in the hydrocarbon feedstock.

The term “alkyne” refers to any hydrocarbon molecule containing at least one triple carbon-carbon bond which can be of aliphatic or cyclic structure, Typically for the present invention, C4 to C10 alkyne may be present in the hydrocarbon feedstock.

The term “nitrogen-containing compound” refers to any compound containing at least one nitrogen atom. Preferably for the invention, the nitrogen compound is an organic nitrogen-containing compound, more preferably N-Formyl Morpholine.

As used herein, the term “montmorillonite” refers to a hydrated aluminium silicate clay mineral with expandable structure consisting of two tetrahedral layers of silica sandwiching a central octahedral layer of alumina. Chemically, it is hydrated sodium calcium aluminium magnesium silicate hydroxide. Potassium, iron, and other cations are possible substitutes, and the ratio of cations can be varied. Between layers, there is free-space that is capable to keep the water, cation, or other adsorbates.

Preferably, contacting the hydrocarbon feedstock with the acidic montmorillonite adsorbent is carried out at a gauge pressure in the range from 0 to 6×10⁶ Pascal, preferably 1 to 1×10⁶ Pascal.

In one embodiment, the acidic montmorillonite adsorbent comprises a content of H⁺ per gram of adsorbent of at least 1 μmol.

The content of H⁺ per gram of adsorbent is the number of exchangeable acid sites per gram of the adsorbent. It can be determined by a method involving an aqueous ion exchange of the adsorbent H+ ions with selected cation, followed by titration of the resulting solution.

Particularly, the content of H⁺ per gram of adsorbent according to the present invention can be determined by the following steps.
(1) Adding 0.2 g of the acidic montmorillonite adsorbent to 10 mL of aqueous solution of 3.4 mol/L NaCl under stirring to perform ion exchange at room temperature;
(2) After 30 hours of ion exchange, solid particle was filtered off;
(3) The filtrate was titrated with 0.05 mol/L of aqueous NaOH solution. The end point of titration (pH is approximately 7) was determined using a pH meter;
(4) The number of exchangeable acid sites in micromol of H+ per gram of adsorbent (μmol H⁺/g-adsorbent) was calculated by the following equation (1)

μmol H⁺/g-adsorbent=10^{3×}(volume of NaOH×concentration of NaOH)/g-adsorbent (1)

Details of the steps of determining the content of H⁺ per gram of adsorbent described above such as amount of adsorbent, type and concentration of ion-exchange solution, type and concentration of titrant could be modified, or even a different method capable of determining content of H⁺ per gr; of adsorbent known to those with ordinary skill in the art could be used without limitation.

Preferably, for this embodiment, the acidic montmorillonite adsorbent having a content of H⁺ per gram of adsorbent of at least 1 μmol is obtained by treating a montmorillonite with an acid.

The acid used for treating the montmorillonite can be any chemical with a pH lower than 7 and is preferably in the form of a solution. Further preferred, the acid is selected from the group consisting of ammonium sulfate, sulfuric acid, hydrochloric acid and mixtures thereof, more preferably ammonium sulfate. Also preferably, the acid has a concentration in the range from 0.5 to 20 percent by weight with respect to the amount of total composition of the solution.

It is preferred that treating the montmorillonite with the acid is performed by contacting the montmorillonite with the acid at a temperature in the range 10 to 60° C., preferably 15 to 35° C.

In another embodiment, the acidic montmorillonite adsorbent is characterized by having at least one NH₃-TPD desorption peak with the size in the range from 9 to 60 ml/g, preferably 20 to 60 ml/g, at a high desorption region of a temperature in the range of 300 to 800° C.

NH₃-TPD is a method that is extensively used to quantify acid sites of a solid material. Typically, the method involves saturating surface of the solid material with NH₃ under certain conditions, followed by linear ramp of temperature in a flowing inert gas stream or connected to vacuum. At higher temperature ammonia is desorbed and the amount of ammonia desorbed is a measure of acid sites on the solid material surface.

Particularly for this invention, the NH₃-TPD desorption peak is determined by carrying out NH₃-Temperature Programmed Desorption (NH₃-TPD) using a TCD detector. Before adsorption, 0.05 g of the acidic montmorillonite adsorbent is dried in a flow of H₂ at 425° C. for 2 hours. Adsorption of 10% NH₃/He then took place at 50° C. until saturation, and then the adsorbent is flushed with He at the same temperature for 2 hours. TCD measurement was carried out from 50 to 800° C. with a heating rate of 10° C./min using He as a carrier gas. Size of the NH₃-TPD desorption peak is calculated from the area of peak.

Preferably for this embodiment, the acidic montmorillonite adsorbent having at least one NH₃-TPD desorption peak with the size in the range of 9 to 60 ml/g at a temperature in the range of 300 to 800° C. is an iron-containing montmorillonite. Further preferably, the iron-containing montmorillonite contains Fe₂O₃ content in a range from 2 to 20 percent by weight, more preferably 8 to 15 percent by weight with respect to the total weight of the adsorbent.

The iron-containing montmorillonite can be naturally occurred or can be synthesized by ion-exchanging a non-iron montmorillonite with iron containing salt.

In another preferred embodiment, the acidic montmorillonite adsorbent according to the present invention is characterized by having a content of H⁺/g-adsorbent of at least 1 μmol and having at least one NH₃-TPD desorption peak with the size in the range of 9 to 60 ml/g, preferably 20 to 60 ml/g at a temperature in the range of 300 to 800° C.

For this embodiment, the acidic montmorillonite adsorbent characterized by having a content of H⁺/g-adsorbent of at least 1 μmol and having at least one NH₃-TPD desorption peak with the size in the range of 9 to 60 ml/g, preferably 20 to 60 ml/g at a temperature in the range of 300 to 800° C. is preferably obtained by treating an iron-containing montmorillonite with an acid. More preferably, the acidic montmorillonite adsorbent characterized by having a content of H⁺/g-adsorbent of at least 1 μmol and having at least one NH₃-TPD desorption peak with the size in the range of 9 to 60 ml/g, preferably 20 to 60 ml/g at a temperature in the range of 300 to 800° C. is obtained by treating the iron-containing montmorillonite containing Fe₂O₃ content in a range from 2 to 20 percent by weight, even more preferably 8 to 15 percent by weight with respect to the total weight of the montmorillonite with an acid selected from the group consisting of ammonium sulfate, sulfuric acid, hydrochloric acid and mixtures thereof, more preferably ammonium sulfate.

Treating the iron-containing montmorillonite with the acid is preferably carried out in the manner already described above.

Alternatively, the acidic montmorillonite adsorbent characterized by having a content of H⁺/g-adsorbent of at least 1 μmol and having at least one NH₃-TPD desorption peak with the size in the range of 9 to 60 ml/g, preferably 20 to 60 ml/g, at a temperature in the range of 300 to 800° C. may also obtained by treating a montmorillonite with an acid first and followed by ion-exchanging the acid treated montmorillonite with Fe-containing salt.

Surprisingly, it was found that the acidic montmorillonite adsorbent according to the invention can selectively adsorb alkene and/or alkyne from a hydrocarbon feedstock comprising an aromatic compound at mild temperature, i.e. below 60° C.

It was also observed that a nitrogen-containing compound contaminant was significantly removed from the hydrocarbon feedstock comprising an aromatic compound by the inventive process.

It was further surprisingly found that, the inventive process does not cause the formation of any by-product and no loss of aromatic compound in the hydrocarbon feedstock was observed.

Preferably, contacting the hydrocarbon feedstock with the acidic montmorillonite adsorbent is carried out at a weight hourly space velocity in the range from 0.01 to 30 hour⁻, more preferably from 0.1 to 15 hour⁻¹, and even more preferably from 1 to 7 hour⁻¹,

The inventive process can further comprise, after contacting the hydrocarbon feedstock with the acidic montmorillonite adsorbent, a step of regenerating the acidic montmorillonite adsorbent which is deactivated or saturated by the adsorbed alkene and/or alkyne. The step of regenerating is carried out by contacting the deactivated acidic montmorillonite adsorbent with an inert gas at a temperature in the range from 100 to 400° C., preferably 150 to 300° C., and more preferably 190 to 200° C.

Preferably, the inert gas used in the step of regenerating is selected from nitrogen, helium, and argon, preferably nitrogen.

Due to mild condition employed by the inventive process, it was found that the adsorbent can easily be regenerated to remove polymer and/or carbon deposited on the surface thereof without destroying its structure. This provides the further advantage of producing much lower solid waste of used adsorbent generated per year.

The invention is now further illustrated on the basis of Examples from which further features and advantages may be taken. It is to be noted that the following explanations are presented for the purpose of illustrating only; they are not intended to be exhaustive or to limit the invention to the precise form disclosed.

### EXAMPLE

The following adsorbents were applied in the below examples;
Adsorbent “A” is a commercially available acid-treated montmorillonite adsorbent according to the invention.
Adsorbent “B” is an unmodified natural montmorillonite adsorbent (comparative example).
Adsorbent “C” is an unmodified natural acidic montmorillonite adsorbent according to the invention.
Adsorbent “D” is an acid treated montmorillonite adsorbent obtained by impregnation of 2 wt % of (NH₄)₂SO₄ over the Adsorbent “C” following by drying at 120° C. for 3 hours, according to the invention.

Characterization properties of each adsorbent are shown in the following Table 1.

TABLE 1

| | Characterization Property | | | | |
|---|---|---|---|---|---|
| | NH3-TPD Desorption | NH3-TPD Desorption | | | |
| | peak size at low temp. | peak size at high temp. | H+/g- | | Fe₂O₃ |
| | (below 300° C.) [ml/g at | (below 300-800° C.) | adsorbent | | content |
| Adsorbent | STP] | [ml/g at STP] | [μmol] | pH | [% wt] |
| A | >43.1 | <8.7 | >500 | 3.3 | 1.56 |
| B | >15.7 | <16.6 | <1 | 0 | 1.6 |
| C | >14.6 | >41.4 | <1 | 0 | 12 |
| D | >30.2 | >33.5 | >50 | 4.3 | 7.3 |

### Example 1

### Removing Cyclopentene from Benzene

Each sample adsorbent was placed into the oven in order to remove trace of water at temperature 180° C. for overnight before testing.

The hydrocarbon feedstock used for testing was 150 ppm of cyclopentene in 0.05 of benzene.

Each adsorbent (previously dried) was added into the 25-mL of the hydrocarbon feedstock and was continuously stirred for 9 hours.

The resulting cyclopentene/benzene sample was then analyzed by Gas Chromatography—Flame Ionization Detector (GC-FID), which is a very co on analytical technique that is widely used in the petrochemical to see the amount of cyclopentene left in the benzene sample.

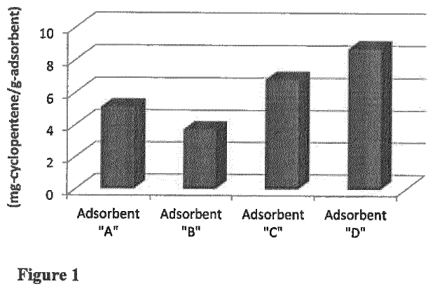

Results of adsorption test for Adsorbent “A”, “B”, “C”, d “D” are displayed in FIG. 1.

### Example 2

The Adsorbent “A”, “B”, “C”, and “D” were tested for adsorption of nitrogen compound. The test was perform by packing 20 g of each adsorbent, previously dried at 180° C. in oven overnight, into separate cylinder vessels. The industrial benzene feedstock, con ng N-Formyl Morpholine (NFM) as a representative of a nitrogen compound, was then flowed pass each of the packed adsorbent at room temperature and ambient pressure. The inlet and outlet benzene concentration were analyzed by GC-FID.

Result of this test is shown in Table 2.

TABLE 2

| | Inlet NFM [ppb] | Outlet NFM [ppb] |
|---|---|---|
| Adsorbent A | 1020 | 104 |
| Adsorbent B | | 332 |
| Adsorbent C | | 16 |
| Adsorbent D | | Non detectable |

From the result above, it can be seen that the inventive adsorbents can also be used for adsorbing a nitrogen compound, which is oftentimes found contaminated in benzene stream.

### Example 3

### Reduction of BI Value in Benzene

The Adsorbent “D” was used to test its efficiency in BI reduction in industrial benzene feedstock. 20 g of Adsorbent “D”, previously dried at 180° C. in an oven overnight, was pack into a cylinder vessel. The industrial benzene feedstock was then flowed pass the packed adsorbent at room temperature and ambient pressure. Inlet and outlet BI value of the industrial benzene were measured according to the standard method ASTM 5776-99. The inlet and outlet benzene concentration were analyzed by GC-FID. The test was repeated 3 times.

Results of this test are displayed in the following Table 2. It can be seen from the results that the Adsorbent “D” can efficiently reduce BI value in benzene feedstock to a very low level. Moreover, no loss of benzene was observed.

TABLE 3

| | 1^{st} Test | | 2^{nd} Test | | 3^{rd} Test | |
|---|---|---|---|---|---|---|
| | Inlet | Outlet | Inlet | Outlet | Inlet | Outlet |
| Bromine Index | 10.10 | 0.4 | 11.55 | 0.9 | 12.87 | 0.5 |
| (BI) Value | | | | | | |
| Benzene | 99.9316 | 99.9532 | 99.9275 | 99.9536 | 99.9063 | 99.9268 |
| (wt %) | | | | | | |

The features described in the foregoing description and in the claims may, both separately and in any combination, be material for realizing the invention in diverse forms thereof.

### Example 4

### Reduction of BI Value in Linear Alkylbenzene (LAB)

The adsorbent “A” and “C” were used to test their efficiency in reduction of BI in linear alkylbenzene.

Feedstock containing linear alkylbenzene (a mixture of linear alkylbenzene with C10-C13 alkyl) and different BI contributors (alkene), i.e., cyclopentene, indene, and 1-undecene, were used.
The testing system was prepared by packing 5 grams of adsorbent in a cylinder vessel, the dry the adsorbent by heating it to 250° C. for 2 hours.
The testing were operated by passing the feedstock through the bed of adsorbent at room temperature (approximately 25-35° C.) and ambient pressure. Inlet and outlet BI values were measured according to the standard method ASTM D 5776-14a.
Test results are shown in Table 4.

TABLE 4

| | | | Outlet BI, | Outlet BI, |
|---|---|---|---|---|
| Aromatic | | | treated by | treated by |
| Compound | BI Contributors | Inlet BI | adsorbent “A” | adsorbent “C” |
| Linear | Cyclopentene | 8.0 | 0.5 | 2.0 |
| alkylbenzene | Indene | 160.0 | 43.0 | 8.5 |
| | 1-Undecene | 8.0 | 2.0 | 0.5 |

1. A process for purifying a hydrocarbon feedstock, said process comprising the steps:
(a) providing the hydrocarbon feedstock comprising an aromatic compound and at least one compound, selected from the group consisting of alkene, alkyne, nitrogen-containing compound or mixtures thereof; and
(b) contacting the hydrocarbon feedstock with an acidic montmorillonite adsorbent at a temperature in the range from 10 to 60° C.

2. The process according to claim 1, wherein the aromatic compound is an aromatic hydrocarbon compound selected from benzene, toluene, xylene, ethylbenzene, their derivatives, and mixtures thereof.

3. The process according to claim 1, wherein the alkene is C4 to C10 alkene.

4. The process according to claim 1, wherein the alkyne is C4 to C10 alkyne.

5. The process according to claim 1, wherein the nitrogen-containing compound is N-formyl morpholine.

6. The process according to claim 1, wherein contacting of the hydrocarbon feedstock with the acidic montmorillonite adsorbent is carried out at a gauge pressure in the range from 0 to 6×10⁶ Pascal.

7. The process according to claim 1, wherein the acidic montmorillonite adsorbent comprises a content of H⁺ per gram of adsorbent of at least 1 μmol.

8. The process according to claim 7, wherein the acidic montmorillonite adsorbent is obtainable by treating a montmorillonite with an acid.

9. The process according to claim 8, wherein the acid is selected from the group consisting of ammonium sulfate, sulfuric acid, hydrochloric acid, and mixtures thereof.

10. The process according to claim 1, wherein the acidic montmorillonite adsorbent is characterized by having at least one NH₃-TPD desorption peak with the size in the range from 9 to 60 ml/g at a high desorption region of a temperature in the range of 300 to 800° C.

11. The process according to claim 10, wherein the acidic montmorillonite adsorbent is an iron-containing montmorillonite.

12. The process according to claim 11, wherein the iron-containing montmorillonite has a Fe2O₃ content in a range from 2 to 20 percent by weight with respect to the total weight of adsorbent.

13. The process according to claim 1, wherein the process further comprises, after contacting the hydrocarbon feedstock with the acidic montmorillonite adsorbent, a step of regenerating the acidic montmorillonite adsorbent.

14. The process according to claim 13, wherein the step of regenerating comprises contacting the acidic montmorillonite adsorbent with an inert gas at a temperature in the range from 100 to 400° C.

15. The process according to claim 14, wherein the inert gas is selected from nitrogen, helium, and argon.
